(19) United States
(12) Patent Application Publication
(10) Pub. No.: US 2008/0262484 A1
(43) Pub. Date: Oct. 23, 2008

(54) Motion-controlled laser surface treatment apparatus

(75) Inventors: R. Thomas Hawkins, Aloha, OR (US); Zhe Huang, Vancouver, WA (US); Jason N. Farmer, Vancouver, WA (US); Scott H. Keeney, Portland, OR (US)

Correspondence Address: KLARQUIST SPARKMAN, LLP, 121 SW SALMON STREET, SUITE 1600, PORTLAND, OR, 97204, US

(73) Assignee: nLight Photonics Corporation

(21) Appl. No.: 11/789,366

(22) Filed: Apr. 23, 2007

(51) Int. Cl. A61B 18/20

(52) U.S. Cl. 606/12; 606/11; 606/9; 607/89

(57) ABSTRACT

An exposure apparatus for skin treatment or other applications includes a laser diode module controller that is configured to operate one or more laser diodes that are situated to provide optical radiation to an exposure aperture. A two-dimensional position sensor is secured to the exposure aperture and provides an exposure aperture translation signal that is coupled to the laser diode module controller. Delivery of optical radiation to the exposure aperture by the laser diode module controller is based on an exposure aperture translation or velocity that is estimated based on the exposure aperture translation signal. In addition, a clock or timer is coupled to the laser diode module controller to permit selection of laser diode pulse duty cycle or to provide safe or comfortable skin treatment. The two dimensional position sensor can be based on optical sensing such as provided in an optical mouse, and can also provide proximity sensing for safe, efficient operation.

### FIELD

The disclosure pertains to laser-based surface treatment apparatus suitable for hair removal and other applications

### BACKGROUND AND SUMMARY

Lasers are used in dermatology for various applications including wrinkle reduction, amelioration of skin discolorations, and hair removal. For such applications, control of the optical energy delivery to the skin can be critical, especially the rate of energy deposition and total energy delivered, to assure safe and effective treatment. Various methods of laser treatment have been developed, typically based on either application of single or multiple laser pulses, or continuous wave laser emission. Systems in which one or more laser pulses are provided to a stationary treatment surface during exposure can be referred to as “single shot” systems while systems intended for exposure while the laser is moved across the treatment surface can be referred to as “repetitively pulsed” or “continuous wave” systems.

In single shot laser exposure systems, a user places a laser delivery aperture at a region to be treated and triggers a single pulse or a predetermined pulse sequence. The user then moves the laser delivery aperture to the next area to be treated and triggers one or more laser pulses or pulse sequences again. With such a system, care must be taken to provide contiguous coverage areas and to avoid overexposure due to overlap of treatment areas.

In repetitively pulsed or continuous wave (CW) exposure systems, the user moves the laser delivery aperture across the treatment area while laser energy is delivered through the aperture. The user must move the laser at an appropriate rate, or the laser device must track where energy has been delivered, so that the energy delivered to each skin area being treated is commensurate with therapeutic requirements. Because each laser delivery device will have an upper limit on available laser intensity for delivery to the skin, there is a corresponding upper limit on rate of motion of the laser head over the skin if treatment is to be therapeutic.

Examples of such systems for hair removal are disclosed in Anderson et al., U.S. Pat. No. 5,595,568. Other dermatological uses of laser treatment and associated apparatus are disclosed in Chess, U.S. Pat. No. 5,057,104. Weckwerth et al., U.S. Pat. No. 6,758,845 (hereinafter “Weckwerth”), discloses a laser skin treatment handpiece that provides position determination using indicia provided on a roller attached to the handpiece. Altshuler and Anderson, U.S. Pat. No. 7,077,840 (hereinafter “Altshuler”), discloses determining a scan velocity of a dermatological treatment head and alerting an operator based on the determination. Weckwerth and Altshuler do not disclose, suggest, or recognize the advantages of using a position or motion sensor with two-dimensional measurement capability to provide additional flexibility in moving the laser delivery device and control of laser parameters. One example of improved apparatus that provide such advantages disclosed herein comprises a rotatable ball and sensors for measuring two-dimensional motion based on rotation of the ball against a target surface. Another such apparatus comprises an optical sensor system similar to that in an optical computer mouse.

In addition, while Weckwerth discloses firing laser pulses as the laser delivery system moves over the skin, Weckwerth fails to recognize that successful hair removal may require pulses of sufficient duration such that the laser delivery head would preferably be stationary during the pulse. Moreover, laser pulse repetition rates are also governed by other factors, including heat build-up in the laser delivery device during operation, so that delivery of therapeutic laser pulses does not depend on position alone. As disclosed herein, a trigger can be provided based on one or more motion measurements (position, displacement, and/or speed) or an elapsed time after a previous pulse, pulse sequence, or other exposure to control firing of a pulsed or continuous wave laser, so that the laser is fired when the head has moved a sufficient distance so as to avoid depositing excess energy into any portion of the skin and/or when sufficient time has elapsed so that the laser functions properly.

According to representative examples of the disclosed technology, optical radiation exposure apparatus comprise an exposure aperture configured to couple optical radiation to a treatment surface, and a position sensor fixed with respect to the treatment aperture, wherein the position sensor provides an output signal associated with exposure aperture location, displacement, or speed in at least two dimensions. In some examples, the position sensor includes an optical emitter configured to direct an interrogating optical flux to at least a portion of the treatment surface, and an optical detector situated to receive an optical flux produced in response to the interrogating optical flux, wherein the output signal is based on the optical flux received by the optical detector. The optical emitter can also be configured to deliver the treatment optical radiation to the treatment surface and the exposure aperture.

In some examples, the position sensor includes an LED or laser configured to irradiate at least a portion of the treatment surface, an image sensor, and a lens situated so as to form an image of at least a portion of the treatment surface at the image sensor based on the LED illumination. In further representative examples, the position sensor comprises a sensor processor that produces the exposure aperture location output signal based on a comparison of at least two images formed at the image sensor. In representative embodiments, at least one laser diode and a light guide or other optical system situated to receive optical radiation produced by the laser diode and direct the optical radiation to the exposure aperture are provided. In representative examples, the exposure aperture is a terminal aperture of the light guide.

In further illustrative examples, a laser diode controller is coupled to the position sensor, wherein at least one of laser diode pulse duration or pulse amplitude is selected by the laser diode controller based on the exposure aperture location output signal. In additional examples, the position sensor signal processor is configured to produce a target surface proximity signal based either on illumination from the LED or laser received by the image sensor or a mechanical or other switch into or incorporated into or otherwise associated with the position sensor. In additional examples, the laser diode controller is coupled to the sensor processor so as to initiate laser diode pulses based on the proximity signal. In other embodiments, the laser diode controller is coupled to the sensor processor so as to initiate laser diode pulses based on the exposure aperture location signal.

In additional examples, a laser diode controller is coupled to the position sensor, and configured so as to activate at least one laser diode so that the optical radiation delivered to the treatment surface is therapeutically effective. In some examples, the therapeutically effective optical radiation is provided for hair removal.

Skin treatment exposure controllers comprise an output configured to be coupled to an optical source and an input configured to receive two dimensional position information associated with a target exposure location. A processor is configured to selectively supply an optical source activation signal based on the two-dimensional position information to the output. In some examples, a clock that produces a clock signal associated with an elapsed time is provided, and the processor is configured to selectively supply the optical source activation signal based on the elapsed time. According to other examples, the processor is configured to produce an estimate of a speed of the target exposure location on a treatment surface, and the processor supplies the optical source activation signal based on the estimated speed. In further examples, the processor is configured to receive an indication of target surface displacement from an exposure aperture to selectively inhibit optical source activation based on the indication. In additional representative embodiments, the processor selectively inhibits optical source activation in response to two dimensional information corresponding to an exposure aperture movement that is less than a required displacement of the exposure aperture.

Methods of skin treatment include providing a first optical radiation exposure to a target area at an exposure aperture, translating the target aperture, and generating a exposure aperture translation signal associated with exposure aperture displacement in at least two dimensions. A second optical radiation exposure is applied in response to the exposure aperture translation signal. In some embodiments, methods further comprise inhibiting application of a second optical radiation exposure in response to the exposure aperture translation signal. In other examples, the second optical radiation exposure has at least one of an associated optical pulse duration, pulse repetition rate, pulse intensity, or duty cycle that are selected based on the aperture translation signal. In further embodiments, the second optical radiation exposure is selected to reduce perceived treatment discomfort. In further examples, the second optical radiation exposure is selected based on a thermal limit or other optical source constraint for a selected optical radiation source. In alternative examples, the second optical radiation exposure is selected based on an exposure aperture translation estimated based on the aperture translation signal. In other examples, the second optical radiation exposure is selected in response to an exposure aperture speed estimated based on the exposure aperture translation signal.

These and other features and aspects of the disclosed technology are set forth below with reference to the accompanying drawings.

### BRIEF DESCRIPTION OF THE DRAWINGS

FIG. 1 is a block diagram of a representative skin treatment apparatus configured to deliver optical radiation to an exposure aperture.

FIG. 2 is a perspective view of a dermatological treatment device that can include one or more semiconductor lasers.

FIG. 3 is a sectional view of a portion of a two dimensional position sensor that includes an LED and an image sensor.

FIG. 4 is a block diagram of a representative method of exposing skin to a treatment optical flux based on position and/or motion of an exposure aperture in at least two dimensions.

FIG. 5 is a schematic plan view of a position sensor that includes a jog ball.

### DETAILED DESCRIPTION

As used in this application and in the claims, the singular forms “a,” “an,” and “the” include the plural forms unless the context clearly dictates otherwise. Additionally, the term “includes” means “comprises.” Further, the term “coupled” means electrically, electromagnetically, or optically coupled or linked and does not exclude the presence of intermediate elements between the coupled items.

The described systems, apparatus, and methods described herein should not be construed as limiting in any way. Instead, the present disclosure is directed toward all novel and non-obvious features and aspects of the various disclosed embodiments, alone and in various combinations and sub-combinations with one another. The disclosed systems, methods, and apparatus are not limited to any specific aspect or feature or combinations thereof, nor do the disclosed systems, methods, and apparatus require that any one or more specific advantages be present or problems be solved.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed systems, methods, and apparatus can be used in conjunction with other systems, methods, and apparatus. Additionally, the description sometimes uses terms like “produce” and “provide” to describe the disclosed methods. These terms are high-level abstractions of the actual operations that are performed. The actual operations that correspond to these terms will vary depending on the particular implementation and are readily discernible by one of ordinary skill in the art.

Some representative embodiments described herein include both motion and timing controls on laser emission, a two dimensional motion sensor, and an emission control configured to prevent the exposure of a common skin surface area (or other target surface area) to multiple optical pulses in rapid succession or a continuous optical exposure that otherwise might be associated with skin damage or discomfort. Representative motion sensors can be based on a rotating ball configured as in, for example, a computer pointing device, or optically based motion sensors such as included in optical mice.

In the description of some examples, communication between or among circuit components is based on electrical signals. As used herein, such signals are associated with analog or digital voltages or currents, or data values stored in a memory. Such signals can be communicated serially on a single conductor, or in parallel using a plurality of conductors, or otherwise coupled as needed. In some examples, position sensors or other devices are secured to or spatially fixed with respect to an exposure aperture. As used herein, “aperture” refers to an opening or window through which optical radiation is transmitted as well as the plate, membrane, or other structure in which the opening is defined. Typically, an exposure aperture is situated near a surface to be treated during exposure, but it will be appreciated that an effective exposure aperture can be defined at remote locations, and imaged onto or at the target.

Examples are described below with reference to skin treatment. However, the disclosed methods and apparatus are not limited to skin treatment but can be used for other types of surface treatment such as, for example, annealing, hardening, or cleaning. Optical exposures suitable for surface treatment depend on surface properties and the type of treatment to be provided. For skin treatment and hair removal, suitable exposures are referred to herein as “therapeutically effective.” Therapeutically effective exposure properties depend on, for example, optical wavelength, optical intensity, total optical energy, optical pulse repetition rate, and prior exposure of a target surface area and time elapsed since the prior exposure as well as skin and/or hair properties of the subject treated.

In typical treatment scenarios, optical radiation is delivered to an exposure aperture that is translated in a plane that is substantially parallel to a treatment surface. A position sensor can be provided that produces an output signal associated with exposure aperture position based on one or more exposure aperture positional coordinates, or one or more components of a displacement or velocity of the exposure aperture with respect to the target surface in a plane substantially parallel to the target surface. Thus, as used herein, a position sensor can provide one or more components of position, displacement, or speed and is not limited to providing positional coordinates. Position sensors can also be configured to provide proximity signals or data that are typically associated with a separation of an exposure aperture from the target surface, generally along an axis that is substantially perpendicular to the target surface.

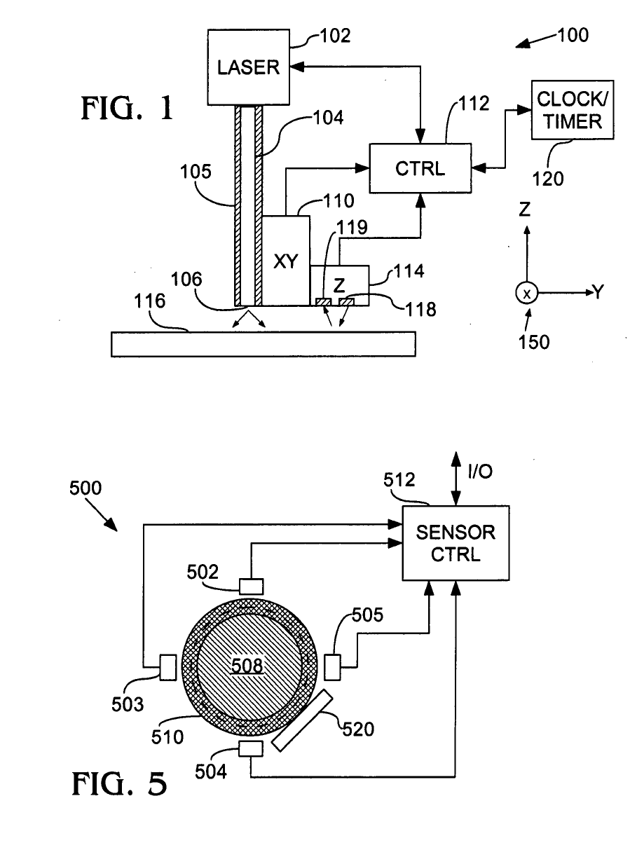

With reference to FIG. 1, an exposure system 100 typically includes a source 102 of laser radiation or other optical source that produces optical radiation that is coupled to a light guide 104 and delivered to an exposure aperture 106. The light guide 104 is typically based on total internal reflection in a section of a solid that is transparent to the optical radiation from the source 102 and may be at least partially enclosed by a sheath 105 that is substantially opaque to optical radiation from the source 102. The sheath 105 can serve to protect the light guide 104 and/or to prevent optical radiation from the source 102 from reaching destinations other than the exposure aperture 106. Convenient light guide materials include glasses and transparent plastics. Alternatively, the light guide 104 can be a hollow light guide in which a light guide cavity is defined by a metallic or other reflective shell. In some examples, the light guide 104 can be configured as a bundle of transparent fibers or based on a flexible metallic shell or tube so that the light guide 104 is flexible. Lens arrays, diffusers, or other light scramblers or randomizers can be provided at a light guide entrance or within a light guide in order to produce a preferred optical radiation distribution at an exit aperture. Typically, the light guide 104 is configured to fill the exposure aperture 106 with a substantially uniform optical radiation, but in other examples, the exposure aperture is only partly filled and the radiation distribution is selectively non-uniform even in filled portions of the exposure aperture 106.

In the example of FIG. 1, the light guide 104 is provided to couple optical radiation to an exposure aperture, but in other examples, one or more lenses or other optical elements can be situated so as to direct optical radiation from the source 102 to the exposure aperture 106 and a light guide is not used. For example, the optical radiation from the source 102 can be focused (or defocused) to provide a selected intensity distribution at the exposure aperture 106. To provide a more uniform intensity at the exposure aperture 106, an optical scrambler can be provided between the source 102 and the exposure aperture 106.

The exposure system 100 also includes a position sensor 110 that is coupled or secured to the light guide 104, the sheath 105, or the exposure aperture 106 to provide coordinates corresponding to a location of the exposure aperture 106. Typically, the position sensor 110 is fixed to the sheath 105 or other support for the light guide 103 and provides position coordinates along orthogonal axes (x- and y-axes in coordinate system 150) that are approximately parallel to a plane containing the exposure aperture 106. Position coordinates can be conveniently provided as analog or digital electric signals. In some examples, the position sensor 110 provides analog coordinates for digitization or other processing by a controller 112. While orthogonal position coordinates are convenient, position coordinates with respect to non-parallel but non-orthogonal axes can be used.

The position sensor 110 can be conveniently provided based on an optical mouse sensor/encoder, either the same as or similar to that of a common optical mouse used with computer systems. Typical target surfaces such as skin provide sufficient texture to provide position coordinates based on estimated displacements in a series of signals associated with two or more target surface areas. Alternatively, a “trackball” type sensor/encoder can be used to provide one or more coordinates. In one example, a spherical ball is configured to roll on the skin or other target surface and to rotate one or more secondary wheels or axles to provide associated coordinates.

The controller 112 is configured to receive analog or digital position coordinates from the position sensor 110 and to initiate, adjust, or regulate operation of the source 102 based on the coordinates from the position sensor 110. Typically the source 102 includes a plurality of laser diodes, and the controller 112 regulates one or more electrical currents supplied to the laser diodes. Depending on the coordinates received from the position sensor 110 or a change in one or more coordinates as determined by the position sensor 110 or determined at the controller 112, the controller 112 can inhibit laser output, increase or decrease laser output power, or control laser pulse duration and/or laser pulse repetition rate or other operational parameters for one or more lasers. A clock 120 (or a timer) is provided, and the controller 112 is configured to initiate or inhibit laser pulses based on an elapsed time. In addition, the controller 112 can be configured to determine an exposure aperture velocity with respect to surface 116 that is to be exposed based on position coordinate data and timing data provided by the clock 120.

A surface proximity sensor 114 is coupled to the controller 112 and provides an position indication or coordinate associated with a separation (along a z-axis as shown in FIG. 1) between the surface 116 that is to be exposed and the exposure aperture 106. In some examples the proximity sensor 114 is configured to provide a position coordinate associated with the displacement and along an axis that is substantially perpendicular to the axes associated with the coordinates provided by the position sensor 110. In other examples, the proximity sensor 114 provides an indication that a predetermined separation has been exceeded and the controller 112 terminates laser emission. In this manner, the laser or other source of optical radiation is active only when the exposure aperture 106 is sufficiently close to a target surface to provide a suitable treatment exposure.

The proximity sensor 114 can be provided as a mechanical switch that is activated or deactivated by skin contact, or an optical proximity sensor can be used. In some examples, an LED is provided as part of an optical-mouse-based position sensor and used to illuminate a portion of the target surface. One or more images of the target surface portion are obtained and position coordinates are based on these images. If the target surface and the LED are sufficiently displaced, then the LED does not efficiently illuminate a selected portion of the target surface (the portion imaged to provide position coordinates), and the magnitude of the detected LED illumination can be used as an indication of target surface/exposure aperture separation. In the example of FIG. 1, the proximity sensor 114 can includes an optical emitter 118 and a detector 119 that are configured so that optical power received at the detector is a function of sensor/target surface displacement. In some convenient examples, both the emitter and the detector can be provided as part of the position sensor 110, and separate or dedicated components are unnecessary.

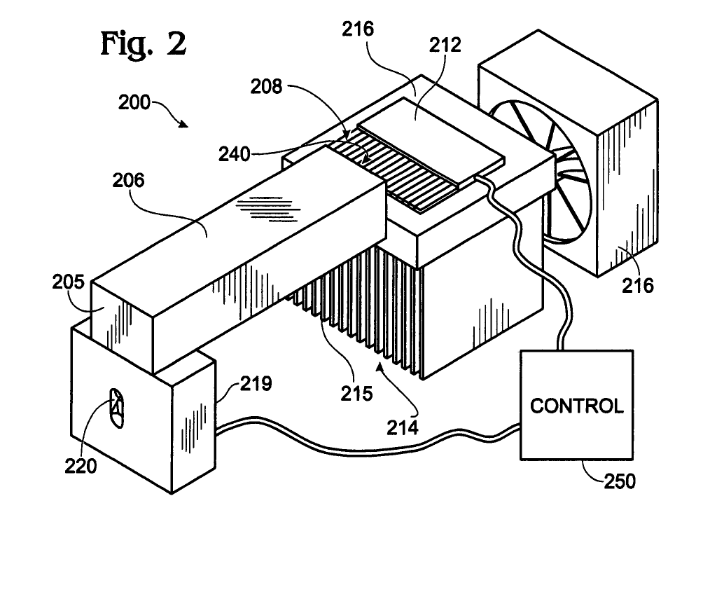

A representative implementation of a laser exposure instrument 200 is illustrated in the perspective view of FIG. 2. The exposure instrument 200 includes an exposure aperture 205 defined by an output face of a light guide 206 that is configured to receive optical radiation produced by a laser diode module 208 that includes one or more laser diodes. The laser diode module 208 is electrically coupled to a circuit board 212 that includes appropriate laser drive circuitry or such circuitry can be provided separately in a controller 250 that is electrically coupled to the circuit board 212. The laser diode module 208 is generally selected to provide optical radiation at a wavelength or in a wavelength range appropriate for an intended treatment application. As used herein, optical radiation includes wavelength in ranges of at least 200-2000 nm, 400-1600 nm, 500-1200 nm, 550-900 nm, and is not necessarily at visible wavelengths. Wavelengths near about 800 nm are particularly suitable for hair removal applications. These and other wavelength ranges are discussed in Weckwerth et al., U.S. Pat. No. 6,758,845 which is incorporated herein by reference. The laser diode module 208 and the circuit board 212 are thermally coupled to a heat sink assembly 214 that includes a support plate 216 and a plurality of fins 215. A fan 216 is situated to produce airflow at the heat sink assembly 214. Typically a cover (not shown in FIG. 2) is provided to protect the laser diode module 208 and to contain stray radiation, and the light guide 206 can be similarly provided with a protective housing.

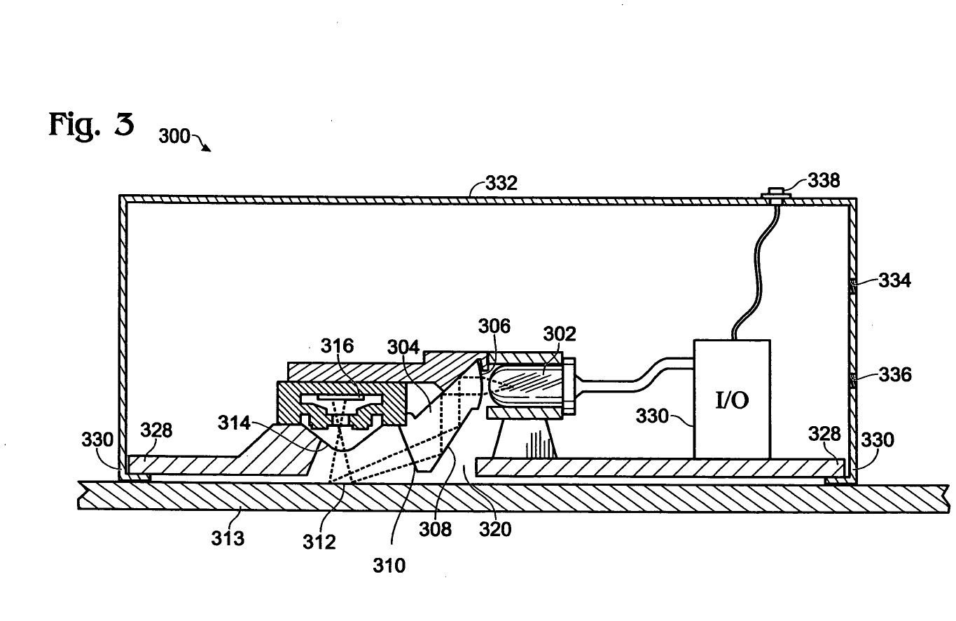

A two dimensional position sensor 219 is fixed to the light guide 206 with a glue layer, screws, a mounting bracket (not shown in FIG. 2), or otherwise secured so that a position sensor aperture 220 is substantially coplanar with the exposure aperture 205. In the example of FIG. 2, the position sensor 219 is similar to that of an optical mouse. The sensor aperture 220 provides optical access to a target surface for both position sensing and proximity detection. The details of such a position sensor are shown in FIG. 3 below. The position sensor 219 is coupled to the controller 250 and provides a position signal associated with position, displacement, speed, or proximity of the exposure aperture 205 with respect to a target surface. The controller 250 is typically configured to process the position signal and to provide power and control signals to the position sensor 219.

The light guide 206 also includes an input face 240 that is configured to receive laser radiation from the laser diode module 208. The input face 240 can be configured based on orientation, number, or distribution of lasers in the laser module 208 or to accommodate additional laser modules. The laser module 208 can be otherwise coupled to the light guide 206 so as to promote a uniform intensity or other intensity distribution at the exposure aperture 205. As shown in FIG. 2, the exposure aperture 205 (the exit face of the light guide 206) and the input face 240 are planar, but in other examples, such surfaces can be spherical, ellipsoidal, cylindrical, or have other shapes or combinations of shapes. The exit aperture can be defined by an external aperture, and need not correspond to the exit surface of a light guide. In some examples, a plurality of segments or lens elements are provided to the input face 240 so as to enhance uniformity at the exposure aperture 205. The light guide 206 can be protected with a light guide housing that can be secured with screws, rivets, glues or otherwise fixed with respect to the laser diode module 208.

A representative position sensor 300 based on an optical mouse is illustrated in FIG. 3. Such optical pointing devices are described in, for example, Leong et al., U.S. Pat. No. 6,967,321 and Gordon et al., U.S. Pat. No. 6,281,882 that are incorporated herein by reference. The sensor 300 includes a light emitting diode (LED) 302 that is secured to a circuit substrate 328 such as a printed circuit board and situated to provide an optical flux to a lens element 304 that includes a convex input surface 306. A portion of the optical flux from the LED 302 is received by the convex surface 306, reflected by an internally reflecting prism surface 308 through an exit surface 310 and a sensor aperture 320 to a target area 312 of treatment specimen 313 such as skin. A portion of the optical flux reaching the target area 312 is generally scattered or reflected to a detector lens 314 that forms an image of the target area 312 or a portion thereof at an image sensor 316.

The position sensor 300 includes a cover 332 to which the circuit substrate 328 is secured. For convenient attachment to a beam delivery light guide or an exposure aperture, threaded holes 334, 336 or other attachment mechanisms are provided. The position sensor 300 also includes an electrical connector 338 configured to communicate electrical signals and power to an input/output (I/O) module 330 to power or otherwise control the LED 302. The I/O module 330 is also coupled to the image sensor 316 with one or more wires or cables, or with conductive traces defined on the circuit substrate 328 so as to receive image or other signals, and to provide any necessary sensor bias or otherwise power or bias the image sensor 316 for operation. Typically, a compact position sensor is preferred so that an exposure aperture and a sensor aperture are close. Processing of image sensor data to produce position coordinates or to determine proximity of the treatment specimen 313 can be provided in the I/O module 330 or can be provided in one or more external processing circuits or circuit assemblies.

In some examples, an image sensor signal is provided by the image sensor 316 to a remote processor in order to capture images and determine position coordinates based on displacements of target area images. In other examples, such a processor and control circuitry are secured to the image sensor, and LED and position coordinates are supplied without additional processing. As shown in FIG. 3, increasing displacements of the target surface 312 and sensor aperture 320 tend to reduce the amount of LED optical flux that reaches the image sensor 316, and the total LED flux received by the image sensor 316 can be used as a proximity signal so that laser diodes modules can be activated or deactivated as intended. In some examples, an associated proximity signal is produced in processing circuitry located within the position sensor 300.

CW or pulsed laser dermatologic exposure and treatment systems such as illustrated in FIGS. 1-2 can provide laser output control based on one or both of timing and exposure aperture movement. With such control, both therapeutic efficacy and skin safety can be improved. At low exposure aperture speeds with respect to a target surface, position and motion sensing can be used so that a laser pulse (or pulse sequence or CW emission) is initiated after the exposure aperture moves far enough so that the exposure aperture is not over an already-treated region of skin. For example, a motion-based laser trigger can be disarmed after the laser is pulsed and then re-armed after the exposure aperture moves far enough for skin safety.

Position and motion based control also offers advantages at higher exposure aperture speeds. A timing trigger can be established so that the laser can be pulsed with a selected pulse intensity and pulse duration without laser overheating as the exposure aperture is moved. Repetitive or single pulse exposure amplitudes, durations, or other parameters can be quantified and selected based on various optical pulse parameters in response to position and timing signals. As used herein, τₚᵤₗₛₑ is an optical pulse duration, Tₚᵤₗₛₑ is an optical pulse period (1/frequency), Wₐₚₑᵣₜᵤᵣₑ is a dimension of the exposure aperture in a direction of motion, and ηₚᵤₗₛₑ=τₚᵤₗₛₑ/Tₚᵤₗₛₑ is a pulse duty cycle. The optical pulse duration τₚᵤₗₛₑ can be a full duration of an optical pulse at a half maximum value or at other values, such as 5%, 10%, or 25%, or an equivalent pulse duration associated with total pulse energy at a peak (or other constant) pulse amplitude. Associated velocities can be defined as V_{I}=Wₐₚₑᵣₜᵤᵣₑ/τₚᵤₗₛₑ and V_{PW}=ηₚᵤₗₛₑWₐₚₑᵣₜᵤᵣₑ/τₚᵤₗₛₑ=ηₚᵤₗₛₑV_{I}.

Various exposure aperture scan rates can be used, and exposure parameters selected to produce safe and effective exposures. In some examples, an optical pulse energy is selected so that a single optical pulse produces an approximately minimum effective treatment exposure with a stationary exposure aperture. Exposure can be initiated based on one, two, or more trigger events. Such trigger events include an elapsed time from a most recent (or other prior) pulse exposure and a predetermined exposure aperture displacement, typically a complete aperture displacement. Upon initiation of a pulse, additional exposures can be disabled until one or both such trigger events occur again. In some examples, the elapsed time can be established to provide skin safety or comfort, or to prevent damage to the optical source from excess heat. Typically, the trigger time can be selected based on a suitable optical source duty cycle. A complete exposure aperture displacement is not necessary, but a trigger displacement can be selected based on appropriate exposure amounts as the exposure aperture moves with respect to the treatment area.

For example, for an exposure aperture speed V, wherein V_{PW}≦V≦V_{I}, adding a timing trigger permits laser activation at full intensity without laser overheating. A timing trigger can be disarmed upon initiation of a laser pulse, and then re-armed after the pulse duration and a subsequent delay (dictated by laser duty factor, skin safety, or other considerations) have elapsed. For example, if the laser pulse duration is 100 ms and the laser duty cycle cannot exceed 40% without risk of overheating, then a duty factor delay between pulses of 150 ms can be provided so that the laser is not on more than 100 ms in any 250 ms period. At still higher velocities V>V_{I}, the combination of motion and timing information can permit a laser controller to either increase laser power (if possible) to maintain therapeutic efficacy or turn off the laser if insufficient laser energy would be delivered to the skin. In other examples, laser power and/or pulse duration can be established to provide eye safety, and motion control used to determine if exposure aperture velocity is too high for effective treatment or if laser power should be adjusted. Such control can be provided with, for example, a digital controller in communication with the laser and that is coupled to receive trigger signals from both a clock and a motion measurement sensor or position sensor. Alternatively, control can be provided based on analog circuit elements such as Schmitt triggers.

Position and motion based laser control is typically provided in order to provide eye safe operation, avoid laser overheating, effective treatment with a moving aperture, or reduce likelihood of skin damage or discomfort. For example, for skin treatment, laser pulses received in a common treatment area within a time period of a few seconds produce a treatment effectiveness associated with the sum of the combined received optical energy. Pulses that are more widely separated in time typically do not provide such a combined effectiveness. Thus, in order to reduce discomfort, a time period of at least a few seconds can be allowed to elapse after treatment of a first area before an additional exposure, while no such delay is needed for exposure of an area that does not overlap the first area. If a position/motion sensor indicates that the exposure aperture has moved sufficiently, the new treatment area is distinct from a previous treatment area and a subsequent treatment can be provided without consideration of the previous treatment. Optical source controllers can thus take advantage of exposure aperture position and velocity.

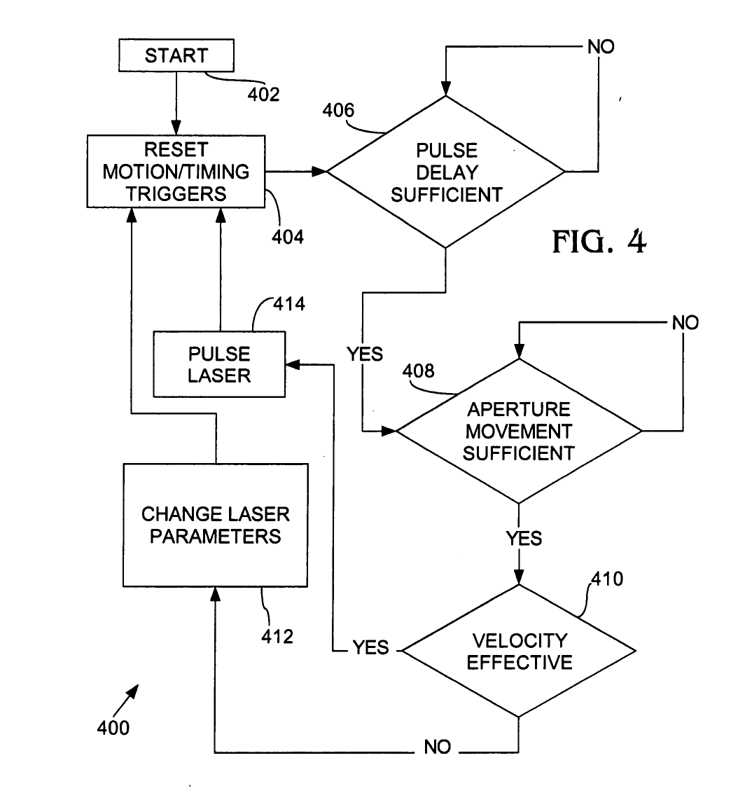

A representative control method 400 for an optical source such as a laser diode in an apparatus such as that of FIG. 1 is illustrated in FIG. 4. In a step 404, motion and timing triggers are reset based on laser pulse durations, skin or other target characteristics, eye safety, pulse rate, or other laser or target considerations. In a step 406, pulse delay is evaluated to determine if the pulse delay is sufficient to provide skin safety or avoid laser overheating. If the pulse delay is sufficient, in a step 408, motion of the exposure aperture is evaluated. This evaluation can be a determination of whether or not the exposure aperture has moved to a different treatment area. In a step 410, exposure aperture velocity is evaluated. If the velocity is suitable for effective treatment, a laser pulse or pulses are initiated in a step 414. If the velocity is unacceptable for effective treatment, laser operation parameters (for example, duration, amplitude, repetition rate, duty cycle) can be changed in a step 412. In some examples, suitable changes to laser parameters are not available due to, for example, eye safety or laser heating concerns, and a warning or notification that effective treatment is not possible under current operating conditions can be made. Upon application of an additional pulse or pulse sequence in the step 414, motion and timing triggers are reset in the step 404 and the above sequence is repeated. With a method such as the method 400, a single skin area is not exposed to multiple pulses within a time window short enough so that skin is damaged and laser operation is within device limits.

While implementations of the disclosed technology with optically based position sensor are convenient, other types of sensors can be used as well. For example, with reference to FIG. 5, a position sensor 500 includes Hall effect sensors 502-505 situated in proximity to a jog ball 508 that is retained by a housing 510 that permits the jog ball 508 to rotate. The Hall effect sensors 502-505 produce electrical signals associated with rotation of the jog ball 508 against a target surface. The position sensor 500 can be secured to a light guide or exposure aperture at a mounting bracket 520 or otherwise fixed with respect to an exposure aperture. A processor 512 is configured to produce one or more electrical signals indicative of position, displacement, or velocity of an exposure aperture based on electrical signals from the Hall effect sensors 502-505 in response to rotation of the jog ball. In other examples, position sensors using a rotatable ball or “track ball” as in FIG. 5 can include other types of encoders to sense ball rotation and Hall effect sensors are not required.

In view of the many possible embodiments to which the principles of the disclosed technology may be applied, it should be recognized that the illustrated embodiments are only illustrative examples and should not be taken as limiting the scope of the disclosure. For example, exposure systems can use optical sources other than lasers, and these sources can provided CW optical fluxes or pulsed optical fluxes, and sources can be either directly pulsed or gated with an external modulator. Position sensors can be combined with optical source controls so as to provide a unitary controller that can also include laser diode modules or other optical sources. Exposure apertures can be square, rectangular, polygonal, circular, elliptical or other shapes, although shapes that can be arranged to cover a surface without overlap are convenient. Position and/or velocity detection can be used to provide optical source control based on displacements or velocity components in one or more directions. While the examples are described above with reference to skin treatment, the disclosed methods and apparatus can be applied to delivery of optical radiation to other surfaces as well. In view of these and other examples, we therefore claim as our invention all that comes within the scope and spirit of the following claims.

We claim:

1. An apparatus, comprising:
an exposure aperture configured to couple optical radiation to a treatment surface; and
a position sensor fixed with respect to the treatment aperture, the sensor providing an output signal associated with exposure aperture location in at least two dimensions.

2. The apparatus of claim 1, wherein the exposure aperture location signal is associated with a displacement of the exposure aperture.

3. The apparatus of claim 1, wherein the exposure aperture location signal is associated with a speed of the exposure aperture with respect to the treatment surface.

4. The apparatus of claim 1, wherein the position sensor includes:
an optical emitter configured to direct an interrogating optical flux to at least a portion of the treatment surface, and
an optical detector situated to receive an optical flux produced in response to the interrogating optical flux, wherein the exposure aperture location signal is based on the optical flux received by the optical detector.

5. The apparatus of claim 4, wherein the exposure aperture output location signal is associated with a displacement of the exposure aperture with respect to a plane substantially parallel to the treatment surface.

6. The apparatus of claim 4, wherein the exposure aperture location signal is associated with a speed of the exposure aperture with respect to the treatment surface.

7. The apparatus of claim 4, wherein the optical emitter is an LED, the detector is an image sensor, and the position sensor further comprises a lens situated so as to form an image of at least a portion of the treatment surface at the image sensor based on the interrogating optical radiation from the LED, and the exposure aperture signal is based on at least two images formed at the image sensor.

8. The apparatus of claim 7, wherein the position sensor further comprises a sensor processor that produces the exposure aperture location signal based on a comparison of the at least two images formed at the image sensor.

9. The apparatus of claim 7, wherein the exposure aperture location signal is associated with a displacement of the exposure aperture with respect to the treatment surface.

10. The apparatus of claim 7, wherein the exposure aperture location signal is associated with a speed of the exposure aperture with respect to the treatment surface.

11. The apparatus of claim 4, further comprising:
at least one laser diode; and
a light guide situated to receive optical radiation produced by the laser diode and direct the optical radiation to the exposure aperture.

12. The apparatus of claim 11, wherein the exposure aperture is a terminal aperture of the light guide.

13. The apparatus of claim 1, further comprising a laser diode controller coupled to the position sensor, wherein at least one of a laser diode pulse trigger, pulse amplitude, pulse duration, or duty cycle is selected by the laser diode controller based on the exposure aperture location output signal.

14. The apparatus of claim 4, wherein the position sensor processor is configured to produce a target surface proximity signal based on the interrogating optical flux from the optical emitter received by the optical detector.

15. The apparatus of claim 14, further comprising a laser diode controller configured to initiate laser diode pulses, wherein the laser diode controller is coupled to the sensor processor so as to selectively produce laser diode pulses based on the target surface proximity signal.

16. The apparatus of claim 4, further comprising a laser diode controller coupled to the sensor processor and configured to initiate laser diode pulses based on the exposure aperture location signal.

17. The apparatus of claim 1, further comprising a laser diode controller coupled to the position sensor, wherein the laser diode controller is configured to activate at least one laser diode so that the optical radiation delivered to the treatment surface is therapeutically effective.

18. The apparatus of claim 17, wherein the controller is configured to activate the at least one laser diode so that the optical radiation is therapeutically effective for hair removal.

19. A surface treatment exposure controller, comprising:
an output configured to be coupled to an optical source;
an input configured to receive two dimensional position information associated with a target exposure location; and
a processor configured to selectively supply an optical source activation signal to the optical source output based on the two dimensional position information.

20. The controller of claim 19, further comprising a clock that produces a clock signal associated with an elapsed time, wherein the processor is configured to selectively supply the optical source activation signal based on the elapsed time.

21. The controller of claim 20, wherein the processor is configured to produce an estimate of a speed of the target exposure location on a treatment surface, and the processor supplies the optical source activation signal based on the estimated speed.

22. The controller of claim 21, wherein the processor is configured to receive an indication of target surface proximity to an exposure aperture and to selectively inhibit optical source activation based on the indication.

23. The controller of claim 19, wherein the processor is configured to selectively inhibit optical source activation in response to the two dimensional position information associated with an exposure aperture movement that is less than a complete displacement of the exposure aperture.

24. The controller of claim 19, wherein the two-dimensional position information is based on a speed of the target exposure location on a treatment surface.

25. The controller of claim 19, wherein the two dimensional position information is based on a displacement of the target exposure location on a treatment surface in a direction substantially parallel to the treatment surface.

26. The controller of claim 19, wherein the processor is configured to selectively supply the optical source activation signal so as to initiate delivery of therapeutically effective optical radiation to the target exposure location.

27. The controller of claim 19, wherein the processor is configured to supply the optical source activation signal so as to initiate delivery optical radiation that is therapeutically effective for hair removal.

28. A method of surface treatment, comprising:
providing a first optical radiation exposure to a target area at an exposure aperture;
translating the exposure aperture and generating an exposure aperture translation signal associated with exposure aperture displacement in at least two dimensions; and
selectively applying a second optical radiation exposure in response to a predetermined value of the exposure aperture translation signal.

29. The method of claim 28, further comprising inhibiting application of a second optical radiation exposure in response to the exposure aperture translation signal.

30. The method of claim 28, wherein the second optical radiation exposure has at least one of an associated optical pulse trigger, optical pulse duration, pulse repetition rate, pulse intensity, and duty cycle selected based on the exposure aperture translation signal.

31. The method of claim 28, wherein target area is a skin area of a subject to be treated, and the second optical radiation exposure is selected to reduce perceived treatment discomfort.

32. The method of claim 28, wherein the second optical radiation exposure is triggered based on a thermal limit of a selected optical radiation source.

33. The method of claim 28, wherein the second optical radiation exposure is selected based on an exposure aperture translation estimated based on the exposure aperture translation signal.

34. The method of claim 28, wherein the second optical radiation exposure is selected based on an exposure aperture speed estimated based on the exposure aperture translation signal.

35. The method of claim 28, wherein the first optical radiation exposure and the second optical radiation exposure are selected to provide therapeutically effective exposures for skin treatment at the target area.

36. The method of claim 28, wherein the first optical radiation exposure and the second optical radiation exposure are selected to provide therapeutically effective exposures for hair removal at the target area.
